(19) United States
(12) Patent Application Publication
(10) Pub. No.: US 2026/0066136 A1
(43) Pub. Date: Mar. 5, 2026

(54) SYSTEMS AND METHODS FOR MATCHING PATIENT DATA TO CLINICAL STUDY SPONSORS

(71) Applicant: 3PH ALLIANCE, INC., Birmingham, AL (US)

(72) Inventors: Satyanarayanan SESHAN, Cumming, GA (US); Carolyn MILLS, Blountsville, AL (US); Gunther SCHRAPPE, Miami Beach, FL (US)

(21) Appl. No.: 18/823,973

(22) Filed: Sep. 4, 2024

(51) Int. Cl. G16H 50/70; G06F 16/22; G16H 10/60

(52) U.S. Cl. CPC G16H 50/70; G06F 16/2291; G16H 10/60

(57) ABSTRACT

Disclosed embodiments may include a method for matching patient data to clinical study sponsors. The system may receive a first data package comprising one or more first patient profiles comprising a plurality of data elements each associated with a respective patient. A data tag indicating a data type for each data element may be assigned. The first data package and the data tags are stored. Study criteria comprising inclusion and exclusion criteria for a first study are received. Each of the inclusion and exclusion criteria of the first study are mapped to a respective data tag. Based on the mapping and the data tags assigned to each data element, one or more second patient profiles from the first data package are identified that are compatible with the inclusion and exclusion criteria. Each data element associated with the identified one or more second patient profiles is shared with the user device.

The disclosed technology relates to systems and methods for matching patient data to clinical study sponsors. Specifically, this disclosed technology relates to aggregating patient study data in a central depository and providing a platform that allows medical data to be collected once and used more than one medical study when the medical data is determined to be compatible.

### BACKGROUND

As medical studies have grown in sophistication, it has become increasingly important to optimize the allocation of resources such that medical data that is collected once can be used to conduct multiple studies. However, traditional systems and methods for matching patient data to clinical study sponsors typically require a study sponsor to contract with a contract research organization, such as a hospital system or medical research institute, in order to design the study, collect patient data that matches inclusion and exclusion criteria of the study, and complete the requested study using the collected information. Conventional systems store the patient data in a single database and use this data exclusively for the purpose of the study. Upon conclusion of the study, the data is provided to the sponsor of the study and the database is decommissioned. However, the data collected can be relevant to many other studies depending on specified inclusion and exclusion criteria for each study. Conventional systems have not provided a solution that would allow valuable medical patient data to be reused for multiple studies after being initially collected.

Accordingly, there is a need for improved systems and methods for matching patient data to clinical study sponsors. Embodiments of the present disclosure are directed to this and other considerations.

### BRIEF DESCRIPTION OF THE DRAWINGS

Reference will now be made to the accompanying drawings, which are not necessarily drawn to scale, and which illustrate various implementations, aspects, and principles of the disclosed technology. In the drawings:

FIG. 1 is block diagram of an example system that may be used to provide matching patient data to clinical study sponsors, according to an example implementation of the disclosed technology.

FIG. 2 is block diagram of an example Clinical Study Patient Matching System used to provide matching patient data to clinical study sponsors, according to an example implementation of the disclosed technology.

FIG. 3 is a flow diagram illustrating an exemplary method for matching patient data to clinical study sponsors in accordance with certain embodiments of the disclosed technology.

### DETAILED DESCRIPTION

Examples of the present disclosure related to systems and methods for matching patient data to clinical study sponsors. More particularly, the disclosed technology relates to systems and methods for allowing clinical research to be embedded into the clinical practice, allowing data to be collected once and used for several purposes, including clinical research, quality improvement and other compliant uses. Specifically, this disclosed technology relates to (i) having individual databases for each medical institution, (ii) allowing data to be collected once, during either clinical care or for a specific clinical research study, (iii) allow sites the flexibility to collect not only a set of basic data elements, but also to collect additional, customized, data elements, (iv) while at the same time standardizing the data collected, so that (v) it can be compared between different institutions that could (or not) be collecting data in the same format, (v) allowing data to be reutilized, compliantly for a variety of uses, by mapping the type of consent associated with the patient data, (vi) allowing that data to be reutilized on new clinical studies in an effective manner, by mining the database for the inclusion and exclusion criteria of the study and (vii) allowing the sites to make any required changes to their databases, even after a study has been locked or used in a publication, by implementing versioning control and visualization.

a platform that enables patient data for medical studies to be collected once and used for multiple purposes, thereby increasing the efficiency of the clinical data ecosystem.

According to some embodiments consistent with the present disclosure, the disclosed systems and methods allows for data to be collected as needed by hospital research systems for their own use and additionally map their own collected data to studies being designed by various third parties. Each hospital system may utilize their own electronic health record (EHR) database that allows for the labeling of patient data with data tags indicating the type of data being stored. Embodiments of the disclosed systems and methods can detect when a hospital systems tagged data is improperly tagged, identify a more appropriate data tag, and normalize the data by assigning the identified data tag to the data. Embodiments of the disclosed systems and methods can also assign standardized data tags indicating a type of patient data to unlabeled patient data through analysis of the patient data. Embodiments of the disclosed systems and methods also allow for users to collect non-standard patient data. The system may detect non-standard patient data elements, generate a unique data tag, and assign the unique data tag to the non-standard patient data elements, thereby allowing the system to manage both standard data types and non-standard data types. The disclosed systems, upon receiving a data package including patient health data from a hospital system, may identify one or more studies that a portion of the data is compatible with based on inclusion and exclusion criteria rules specified for each study. Further, the disclosed systems may identify patient data that provides a partial study match to a study specified by a study sponsor based on specified inclusion and exclusion criteria, identify one or more additional data elements that, if collected for a particular patient or patient(s), would transform the patient data partial match into a total match, and allow said patient data to be selectively shared with the study sponsor organizations. The disclosed systems and methods are able to autonomously identify both full and partial matches between patient data and medical studies, and allow patient data collected once to be efficiently reused in additional studies based on inclusion and exclusion compatibility criteria, thereby providing participating organizations with enhanced value from their existing patient data, which is a significant technical improvement over prior technologies for matching patient data to medical studies.

Some implementations of the disclosed technology will be described more fully with reference to the accompanying drawings. This disclosed technology may, however, be embodied in many different forms and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods.

Reference will now be made in detail to example embodiments of the disclosed technology that are illustrated in the accompanying drawings and disclosed herein. Wherever convenient, the same reference numbers will be used throughout the drawings to refer to the same or like parts.

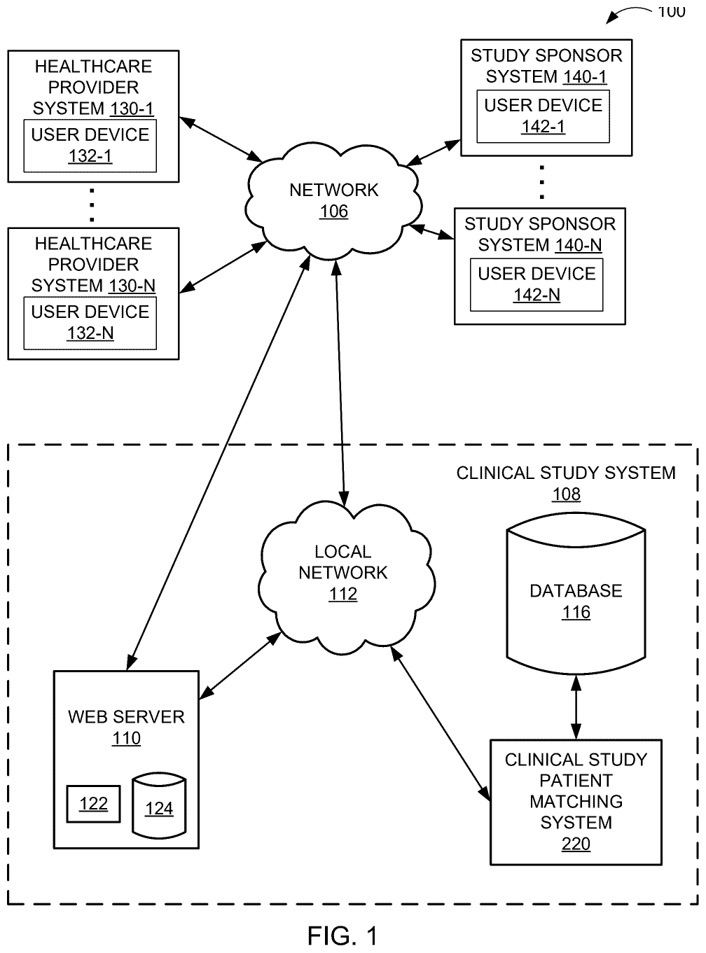

FIG. 1 is a block diagram of an example system that may be used to interact with Clinical Study System 108, according to an example implementation of the disclosed technology. The components and arrangements shown in FIG. 1 are not intended to limit the disclosed embodiments as the components used to implement the disclosed processes and features may vary. As shown, Clinical Study System 108 may interact with one or more healthcare provider systems 130 and one or more study sponsor systems 140 via a network 106. In certain example implementations, the Clinical Study System 108 may include a local network 112, a Clinical Study Patient Matching System 220, a web server 110, and a database 116.

Further, each study sponsor system 140 may have one or more user devices 142 associated with it, that may be used by and/or associated with agents and/or employees of the respective study sponsor system 140. The user device 142 can include one or more of a mobile device, smart phone, general purpose computer, tablet computer, laptop computer, telephone, public switched telephone network (PSTN) landline, smart wearable device, voice command device, other mobile computing device, or any other device capable of communicating with the network 106 and ultimately communicating with one or more components of the Clinical Study System 108. In some embodiments, the user device 142 may include or incorporate electronic communication devices for hearing or vision impaired users. According to some embodiments, the user device 142 may include an environmental sensor for obtaining audio or visual data, such as a microphone and/or digital camera, a geographic location sensor for determining the location of the device, an input/output device such as a transceiver for sending and receiving data, a display for displaying digital images, one or more processors, and a memory in communication with the one or more processors. Similarly to the one or more user devices 142 described above, each healthcare provider system 130-1 may include one or more associated user devices 132 associated with it, that may be used by and/or associated with agents and/or employees of the healthcare provider system 130. User device 130 may have some or all of the components described with respect to user device 140.

The network 106 may be of any suitable type, including individual connections via the internet such as cellular or WiFi networks. In some embodiments, the network 106 may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, WAN, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security.

The network 106 may include any type of computer networking arrangement used to exchange data. For example, the network 106 may be the Internet, a private data network, virtual private network (VPN) using a public network, and/or other suitable connection(s) that enable(s) components in the system 100 environment to send and receive information between the components of the system 100. The network 106 may also include a PSTN and/or a wireless network.

The Clinical Study System 108 may be associated with and optionally controlled by one or more entities such as a business, corporation, individual, partnership, or any other entity that provides one or more of goods, services, and consultations to individuals such as customers. In some embodiments, the Clinical Study System 108 may be controlled by a third party on behalf of another business, corporation, individual, partnership. The Clinical Study System 108 may include one or more servers and computer systems for performing one or more functions associated with products and/or services that the organization provides.

Web server 110 may include a computer system configured to generate and provide one or more websites accessible to users of clinical study system 108, as well as any other individuals involved in system 108's normal operations. Web server 110 may include a computer system configured to receive communications from healthcare provider system 130, study sponsor system 140, and/or user device 142 via for example, a mobile application, a program, an application programming interface, or any other type or format of electronic communication. Web server 110 may have one or more processors 122 and one or more web server databases 124, which may be any suitable repository of website data. Information stored in web server 110 may be accessed (e.g., retrieved, updated, and added to) via local network 112 and/or network 106 by one or more devices or systems of system 100. In some embodiments, web server 110 may host websites or applications that may be accessed by healthcare provider system 130, study sponsor system 140, and/or user device 142. For example, web server 110 may host a web platform for sharing patient data (e.g., by one or more healthcare provider systems 130) on one hand, and specifying medical studies including inclusion and exclusion criteria (e.g., by one or more study sponsor systems 140). Users of clinical study system 108 may gain access by providing an attempted login that are authenticated by the Clinical Study Patient Matching System 220. The web server may also be hosted by an online provider of website hosting, networking, cloud, or backup services, such as Microsoft Azure™ or Amazon Web Services™.

The local network 112 may include any type of computer networking arrangement used to exchange data in a localized area, such as WiFi, Bluetooth™, Ethernet, and other suitable network connections that enable components of the Clinical Study System 108 to interact with one another and to connect to the network 106 for interacting with components in the system 100 environment. In some embodiments, the local network 112 may include an interface for communicating with or linking to the network 106. In other embodiments, certain components of the Clinical Study System 108 may communicate via the network 106, without a separate local network 112.

The Clinical Study System 108 may be hosted in a cloud computing environment (not shown). The cloud computing environment may provide software, data access, data storage, and computation. Furthermore, the cloud computing environment may include resources such as applications (apps), VMs, virtualized storage (VS), or hypervisors (HYP). Healthcare provider system 130, study sponsor system 140, and/or user device 142 may be able to access Clinical Study System 108 using the cloud computing environment. Healthcare provider system 130, study sponsor system 140, and/or user device 142 may be able to access Clinical Study System 108 using specialized software. The cloud computing environment may eliminate the need to install specialized software on healthcare provider system 130, study sponsor system 140, and/or user device 142.

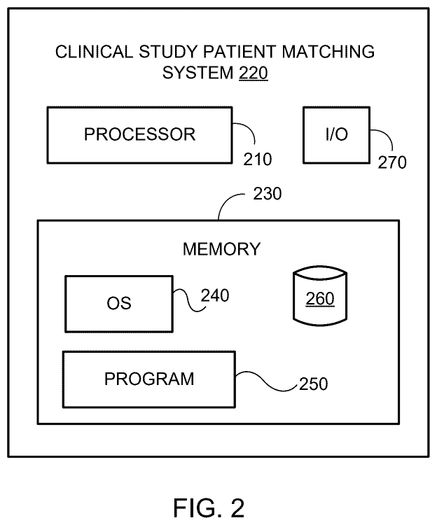

In accordance with certain example implementations of the disclosed technology, the Clinical Study System 108 may include one or more computer systems configured to compile data from a plurality of sources including the Clinical Study Patient Matching System 220, web server 110, and/or the database 116. The Clinical Study Patient Matching System 220 may correlate compiled data, analyze the compiled data, arrange the compiled data, generate derived data based on the compiled data, and store the compiled and derived data in a database such as the database 116. According to some embodiments, the database 116 may serve as a back-up storage device and may contain data and information that is also stored on, for example, database 360, as is discussed below with reference to FIG. 2 and clinical study patient matching system 220. Although database 116 is shown as a single component, it should be understood that database 116 can be implemented as a plurality of databases, a cloud service, etc. In some embodiments, a separate database 116 can be maintained for each entity that chooses to share data with Clinical Study Patient Matching System 220. In other embodiments, a single database 116 is maintained to store the data.

Embodiments consistent with the present disclosure may include datasets. Datasets may comprise actual data reflecting real-world conditions, events, and/or measurements. Datasets may include patient health data including treatments, conditions, outcomes, biometrics, and any other medical information that may be registered and recorded by healthcare provider system 130, study sponsor system 140, and/or user device 142. Datasets of the embodiments may be in a variety of data formats including, but not limited to, PARQUET, AVRO, SQLITE, POSTGRESQL, MYSQL, ORACLE, HADOOP, CSV, JSON, PDF, JPG, BMP, and/or other data formats.

Datasets of disclosed embodiments may have a respective data schema (e.g., structure), including a data type, key-value pair, label, metadata, field, relationship, view, index, package, procedure, function, trigger, sequence, synonym, link, directory, queue, or the like. Datasets of the embodiments may contain foreign keys, for example, data elements that appear in multiple datasets and may be used to cross-reference data and determine relationships between datasets. Foreign keys may be unique (e.g., a personal identifier) or shared (e.g., a postal code). Datasets of the embodiments may be “clustered,” for example, a group of datasets may share common features, such as overlapping data, shared statistical properties, or the like. Clustered datasets may share hierarchical relationships (e.g., data lineage).

Although the preceding description describes various functions of a web server 110, a Clinical Study Patient Matching System 220, a database 116, in some embodiments, some or all of these functions may be carried out by a single computing device.

FIG. 2 is a block diagram of an example Clinical Study Patient Matching System 220 used to determine matches between patient medical data and one or more medical studies, according to an example implementation of the disclosed technology. According to some embodiments, healthcare provider system 130, study sponsor system 140, user device 142, and web server 110, as depicted in FIG. 1 and described above, may have a similar structure and components that are similar to those described with respect to Clinical Study Patient Matching System 220 shown in FIG. 2. As shown, the Clinical Study Patient Matching System 220 may include a processor 210, an input/output (I/O) device 270, a memory 230 containing an operating system (OS) 240 and a program 250. In certain example implementations, the Clinical Study Patient Matching System 220 may be a single server or may be configured as a distributed computer system including multiple servers or computers that interoperate to perform one or more of the processes and functionalities associated with the disclosed embodiments. In some embodiments Clinical Study Patient Matching System 220 may be one or more servers from a serverless or scaling server system. In some embodiments, the Clinical Study Patient Matching System 220 may further include a peripheral interface, a transceiver, a mobile network interface in communication with the processor 210, a bus configured to facilitate communication between the various components of the Clinical Study Patient Matching System 220, and a power source configured to power one or more components of the Clinical Study Patient Matching System 220.

A peripheral interface, for example, may include the hardware, firmware and/or software that enable(s) communication with various peripheral devices, such as media drives (e.g., magnetic disk, solid state, or optical disk drives), other processing devices, or any other input source used in connection with the disclosed technology. In some embodiments, a peripheral interface may include a serial port, a parallel port, a general-purpose input and output (GPIO) port, a game port, a universal serial bus (USB), a micro-USB port, a high-definition multimedia interface (HDMI) port, a video port, an audio port, a Bluetooth™ port, a near-field communication (NFC) port, another like communication interface, or any combination thereof.

In some embodiments, a transceiver may be configured to communicate with compatible devices and ID tags when they are within a predetermined range. A transceiver may be compatible with one or more of: radio-frequency identification (RFID), near-field communication (NFC), Bluetooth™, low-energy Bluetooth™ (BLE), WiFi™, ZigBee™, ambient backscatter communications (ABC) protocols or similar technologies.

A mobile network interface may provide access to a cellular network, the Internet, or another wide-area or local area network. In some embodiments, a mobile network interface may include hardware, firmware, and/or software that allow(s) the processor(s) 210 to communicate with other devices via wired or wireless networks, whether local or wide area, private or public, as known in the art. A power source may be configured to provide an appropriate alternating current (AC) or direct current (DC) to power components.

The processor 210 may include one or more of a microprocessor, microcontroller, digital signal processor, co-processor or the like or combinations thereof capable of executing stored instructions and operating upon stored data. The memory 230 may include, in some implementations, one or more suitable types of memory (e.g. such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system, application programs (including, for example, a web browser application, a widget or gadget engine, and or other applications, as necessary), executable instructions and data. In one embodiment, the processing techniques described herein may be implemented as a combination of executable instructions and data stored within the memory 230.

The processor 210 may be one or more known processing devices, such as, but not limited to, a microprocessor from the Core™ family manufactured by Intel™, the Ryzen™ family manufactured by AMD™, or a system-on-chip processor using an ARM™ or other similar architecture. The processor 210 may constitute a single core or multiple core processor that executes parallel processes simultaneously, a central processing unit (CPU), an accelerated processing unit (APU), a graphics processing unit (GPU), a microcontroller, a digital signal processor (DSP), a field-programmable gate array (FPGA), an application-specific integrated circuit (ASIC) or another type of processing component. For example, the processor 210 may be a single core processor that is configured with virtual processing technologies. In certain embodiments, the processor 210 may use logical processors to simultaneously execute and control multiple processes. The processor 210 may implement virtual machine (VM) technologies, or other similar known technologies to provide the ability to execute, control, run, manipulate, store, etc. multiple software processes, applications, programs, etc. One of ordinary skill in the art would understand that other types of processor arrangements could be implemented that provide for the capabilities disclosed herein.

In accordance with certain example implementations of the disclosed technology, the Clinical Study Patient Matching System 220 may include one or more storage devices configured to store information used by the processor 210 (or other components) to perform certain functions related to the disclosed embodiments. In one example, the Clinical Study Patient Matching System 220 may include the memory 230 that includes instructions to enable the processor 210 to execute one or more applications, such as server applications, network communication processes, and any other type of application or software known to be available on computer systems. Alternatively, the instructions, application programs, etc. may be stored in an external storage or available from a memory over a network. The one or more storage devices may be a volatile or non-volatile, magnetic, semiconductor, tape, optical, removable, non-removable, or other type of storage device or tangible computer-readable medium.

The Clinical Study Patient Matching System 220 may include a memory 230 that includes instructions that, when executed by the processor 210, perform one or more processes consistent with the functionalities disclosed herein. Methods, systems, and articles of manufacture consistent with disclosed embodiments are not limited to separate programs or computers configured to perform dedicated tasks. For example, the Clinical Study Patient Matching System 220 may include the memory 330 that may include one or more programs 250 to perform one or more functions of the disclosed embodiments. For example, in some embodiments, the Clinical Study Patient Matching System 220 may utilize a pre-stored data dictionary to determine which data tag a particular data element being analyzed by Clinical Study Patient Matching System 220 should be assigned.

The processor 210 may execute one or more programs 250 located remotely from the Clinical Study Patient Matching System 220. For example, the Clinical Study Patient Matching System 220 may access one or more remote programs that, when executed, perform functions related to disclosed embodiments.

The memory 230 may include one or more memory devices that store data and instructions used to perform one or more features of the disclosed embodiments. The memory 230 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The memory 230 may include software components that, when executed by the processor 210, perform one or more processes consistent with the disclosed embodiments. In some embodiments, the memory 230 may include a Clinical Study Patient Matching System database 260 for storing related data to enable the Clinical Study Patient Matching System 220 to perform one or more of the processes and functionalities associated with the disclosed embodiments.

The Clinical Study Patient Matching System database 260 may include predetermined data tags related to standard data types for various medical studies. According to some embodiments, the functions provided by the Clinical Study Patient Matching System database 260 may also be provided by a database that is external to the Clinical Study Patient Matching System 220, such as the database 116 as shown in FIG. 1.

The Clinical Study Patient Matching System 220 may also be communicatively connected to one or more memory devices (e.g., databases) locally or through a network. The remote memory devices may be configured to store information and may be accessed and/or managed by the Clinical Study Patient Matching System 220. By way of example, the remote memory devices may be document management systems, Microsoft™ SQL database, SharePoint™ databases, Oracle™ databases, Sybase™ databases, or other relational or non-relational databases. Systems and methods consistent with disclosed embodiments, however, are not limited to separate databases or even to the use of a database.

The Clinical Study Patient Matching System 220 may also include one or more I/O devices 270 that may comprise one or more interfaces for receiving signals or input from devices and providing signals or output to one or more devices that allow data to be received and/or transmitted by the Clinical Study Patient Matching System 220. For example, the Clinical Study Patient Matching System 220 may include interface components, which may provide interfaces to one or more input devices, such as one or more keyboards, mouse devices, touch screens, track pads, trackballs, scroll wheels, digital cameras, microphones, sensors, and the like, that enable the Clinical Study Patient Matching System 220 to receive data from a user (such as, for example, via the user device 142).

In examples of the disclosed technology, the Clinical Study Patient Matching System 220 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

Furthermore, the Clinical Study Patient Matching System 220 may include programs configured to retrieve, store, and/or analyze properties of data models and datasets. For example, Clinical Study Patient Matching System 220 may include or be configured to implement one or more data-profiling models. A data-profiling model may include machine learning models and statistical models to determine the data schema and/or a statistical profile of a dataset (e.g., to profile a dataset), consistent with disclosed embodiments. A data-profiling model may include an RNN model, a CNN model, or other machine-learning model. The Clinical Study Patient Matching System 220 may include algorithms to determine a data type and assign a relevant predetermined data tag to each data element based on the determined data type. The Clinical Study Patient Matching System 220 may be configured to implement univariate and multivariate statistical methods. The Clinical Study Patient Matching System 220 may include a regression model, a Bayesian model, a statistical model, a linear discriminant analysis model, or other classification model configured to determine one or more descriptive metrics of a dataset. For example, Clinical Study Patient Matching System 220 may include algorithms to determine an average, a mean, a standard deviation, a quantile, a quartile, a probability distribution function, a range, a moment, a variance, a covariance, a covariance matrix, a dimension and/or dimensional relationship (e.g., as produced by dimensional analysis such as length, time, mass, etc.) or any other descriptive metric of a dataset.

While the Clinical Study Patient Matching System 220 has been described as one form for implementing the techniques described herein, other, functionally equivalent, techniques may be employed. For example, some or all of the functionality implemented via executable instructions may also be implemented using firmware and/or hardware devices such as application specific integrated circuits (ASICs), programmable logic arrays, state machines, etc. Furthermore, other implementations of the Clinical Study Patient Matching System 220 may include a greater or lesser number of components than those illustrated.

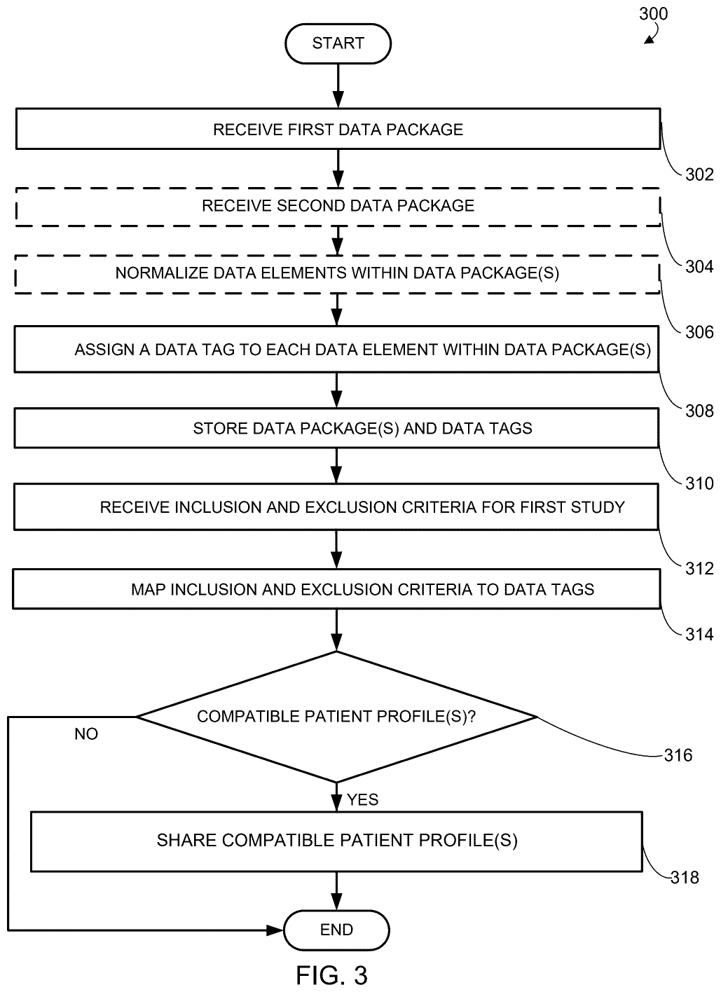

FIG. 3 is a flow diagram illustrating an exemplary method 300 for matching patient data to clinical study sponsors, in accordance with certain embodiments of the disclosed technology. The steps of method 300 may be performed by one or more components of the system 100 (e.g., Clinical Study Patient Matching System 220 or web server 110 of Clinical Study System 108), as described with respect to FIGS. 1 and 2.

In block 302, the Clinical Study Patient Matching System 220 may receive a first data package. The first data package can be received from a healthcare provider system 130 (e.g., healthcare provider system 130-1) or from a user associated with healthcare provider system 130 (e.g., an employee utilizing user device 132-1 that is associated with healthcare provider system 130. The data package can comprise one or more patient profiles. Each patient profile can include a plurality of data elements that are associated with the patient profile, and can include medical information that is collected by healthcare provider system 130 in part of providing healthcare services and treatments to patients. According to some embodiments consistent with the present disclosure, the first data package may be untagged. In other words, the first data package may include data elements that are uncategorized and as such, cannot be directly analyzed to determine compatibility with a given medical study. As used herein, the Clinical Study Patient Matching System 220 receiving a data package can occur in a variety of ways. In one example, the Clinical Study Patient Matching System 220 can directly receive a data package from healthcare provider system 130 and/or study sponsor system 140. In another example, the Clinical Study Patient Matching System 220 can directly receive a data package from a user device 132 or user device 142 associated with healthcare provider system 130 and study sponsor system 140, respectively. In yet another example, data packages can be received by the Clinical Study Patient Matching System 220 via a web portal that is operated by web server 110. The resultant data may be relayed to Clinical Study Patient Matching System 220 via the web server 110. Notably, results can be received via web server from user device 130, user device 140, healthcare provider system 130, and/or study sponsor system 140.

In optional block 304, the Clinical Study Patient Matching System 220 may receive a second data package. The second data package can be received from a second healthcare provider system 130 (e.g., healthcare provider system 130-2). The second data package can comprise one or more patient profiles. Each patient profile can include a plurality of data elements that are associated with the patient profile, and can include medical information that is collected by healthcare provider system 130 in part of providing healthcare services and treatments to patients. According to some embodiments consistent with the present disclosure, the second data package may be tagged. In other words, the second data package may include data elements that have been categorized by healthcare provider system 130-2. In some cases, the data tags that are assigned by the healthcare provider system 130-2 may coincide with standard data tags stored by the Clinical Study Patient Matching System 220, but not necessarily so. In some embodiments, the data tags assigned by healthcare provider system 130-2 may relate to unique data types for which the Clinical Study Patient Matching System 220 does not have standardized data tags. In some embodiments, one or more data tags assigned by healthcare provider system 130-2 may be improperly applied to a particular data element, such that it is improperly labeled.

In optional block 306, the Clinical Study Patient Matching System 220 may normalize the data elements within the data packages received in block 302 and 304. In this regard, the system may parse through each data element within each received data package and determine whether any data tag has been assigned. In the event that data tags are already assigned, as in the case of the data elements within the second data package of block 304, the system can determine if the assigned tags match to the predetermined data tags stored by Clinical Study Patient Matching System 220. In the event that the data tags do not match to the predetermined data tags stored by the Clinical Study Patient Matching System 220, the system may determine identify an appropriate data tag of the stored predetermined data tags, and assign the new data tag to the given data element. This process may be referred to herein as mapping the healthcare provider system tags to the data tags stored by the Clinical Study Patient Matching System 220. In another example, normalization may include determining that an element should be transformed to be compatible with the stored data tag of the Clinical Study Patient Matching System 220. In this regard, one of the data elements received in the data package by Clinical Study Patient Matching System 220 may be in a format of “Age of Patient in Months.” However, the data endpoint that may be required as inclusion criteria for a study may be that the patient Age should be calculated in years. Accordingly, Clinical Study Patient Matching System 220 may transform the data element from “age in months” to “age in years” and apply the appropriate data tag to the transformed data element.

In block 308, the Clinical Study Patient Matching System 220 may assign to each data element an appropriate predetermined data tag that indicates a data type for each data element. In block 310, the Clinical Study Patient Matching System 220 may store each of the plurality of data entries of the received data packages in addition to the data tags assigned to each data element.

In block 312, the Clinical Study Patient Matching System 220 may receive study criteria for a first study from a study sponsor system 140 (e.g., from first study sponsor system 140-1). In this regard, an associate of the first study sponsor system 140-1 (e.g., via user device 142-1 associated with the associate) may transmit study criteria to the Clinical Study Patient Matching System 220. The study criteria may include both inclusion criteria for the inclusion for a given patient's medical data into the first study as well as exclusion criteria for a given patient's medical data to be excluded from the first study.

In block 314, the Clinical Study Patient Matching System 220 may map each of the inclusion and exclusion criteria of the first study to the data tags assigned to each data element within the data package(s) received by the Clinical Study Patient Matching System 220. In this regard, the system checks each of the data elements associated with a given patient profile. If one of the data elements associated with the given patient profile is mapped to an exclusion criteria, the data elements associated with the given patient profile is excluded from the first study. If each of the inclusion criteria is satisfied by at least one of the data elements associated with a given patient profile, the data elements associated with the given patient profile is included in the study. According to some embodiments consistent with the present disclosure, inclusion criteria for a respective study may also include patient consents. For example, before a compatible patient profile is shared with a study sponsor, the respective patient may first provide consent for the data to be shared before the data is made available to the study sponsor system 140. In a similar manner, the healthcare service provider system 130 may first provide consent for the data to be shared before the data is made available to the study sponsor system 140. Finally, inclusion criteria may additionally include consent from the study sponsor system 140. In such a way, consent may be collected from each party associated with the patient data before the data is shared with another party.

In decision block 316, the Clinical Study Patient Matching System 220 may determine whether any of the patient profiles within the data package(s) received by the Clinical Study Patient Matching System 220 are compatible with the first study based on the mapping of the inclusion and exclusion criteria to the data tags. Method 300 may end in response to determining that no patient profiles are compatible with the first study. In block 318, the Clinical Study Patient Matching System 220 may share the data elements associated with the compatible patient profiles in response to identifying one or more compatible patient profiles with the first study sponsor system 140-1. Notably, the first study sponsor system 140-1 is given the option to decline or accept any patient profile for inclusion in the study. The Clinical Study Patient Matching System 220 determines compatibility and shares the compatible patient profiles with the study sponsor system 140, and the study sponsor system 140-1 is given the option whether to include or exclude a given patient.

In some embodiments, the Clinical Study Patient Matching System 220 may receive second study criteria form the first study sponsor system 140-1. The second study criteria can comprise inclusion and exclusion criteria for a sub-study of the first study. In this regard, the Clinical Study Patient Matching System 220 can determine whether a subgroup of the one or more patient profiles identified as compatible with the first study criteria are compatible with the inclusion and exclusion criteria of the sub-study. Upon determining that the subgroup of the one or more patient profiles is compatible with the sub-study, the Clinical Study Patient Matching System 220 may share the data elements associated with the identified subgroup with the first study sponsor system 140-1.

In some embodiments, the Clinical Study Patient Matching System 220 may identify a partial match between a given patient's medical profile and a study with given inclusion and exclusion criteria. In this regard, for example, Clinical Study Patient Matching System 220 may determine that each data element associated with a given patient profile satisfies all but one or more of the inclusion criteria and the given patient profile does not include a data element associated with a study exclusion profile. In this instance, the Clinical Study Patient Matching System 220 may identify one or more data tags associated with the “missing” data elements and provide an indication to the healthcare provider system 130 that if the missing data elements are collected for the given patient, the patient profile will be eligible for the study. This allows healthcare provider system 130 to maximize the utility of healthcare data already being collected by increasing the number of studies that the healthcare provider system 130 can share data with.

In some embodiments Clinical Study Patient Matching System 220 may “freeze” the patient profile when the patient profile is shared with the study sponsor system 140. In this regard, if additional patient data elements are collected for a respective patient profile after the data elements of the patient profile are shared with the study sponsor system 140, these later-collected data elements are not included in the data that is shared with the study sponsor system 140. However, Clinical Study Patient Matching System 220 may be configured to separately store a “live” version of the patient profile that includes the additional data elements as they are received by Clinical Study Patient Matching System 220 from one or more external sources, such as healthcare provider system 130.

In some embodiments, Clinical Study Patient Matching System 220 may receive a second study inclusion and exclusion criteria from a second study sponsor system 140 (e.g., study sponsor system 140-2). In this regard, Clinical Study Patient Matching System 220 may perform a similar operation as was described above with respect to FIG. 3 to determine whether the received data package(s) include one or more patient profiles that are compatible with the second study inclusion and exclusion criteria. For example, the Clinical Study Patient Matching System 220 may identify a subgroup of patient profiles that were shared with the first study sponsor system 140-1 are compatible with the inclusion and exclusion criteria of the second study of the second study sponsor system 140-2. In response, the Clinical Study Patient Matching System 220 may share the subgroup of patient profiles with the second study sponsor system 140-2.

In some embodiments, the Clinical Study Patient Matching System 220 may include a stored data dictionary that includes each predefined data tag stored by the Clinical Study Patient Matching System 220 as well as a plurality of rules that Clinical Study Patient Matching System 220 uses to determine the applicability of each stored data tag when assigning data tags to untagged data elements.

In some embodiments, the Clinical Study Patient Matching System 220 is configured to identify data elements for which no stored data tag exists. In response to identifying a data element for which no stored data tag exists, the Clinical Study Patient Matching System 220 may be configured to generate a new, unique data tag and assign the unique data tag to the data element. In this way, Clinical Study Patient Matching System 220 is able to maintain and store clinical endpoints that are outside of data types generally collected for medical patient studies.

### Example Use Case

The following example use case describes an example of a typical user flow pattern. This section is intended solely for explanatory purposes and not in limitation.

In one example a study is specified by a study sponsor system that includes the following inclusion criteria: “collect patient health data variable 1 through variable 20 (e.g., blood pressure, heart rate, electrocardiogram results, etc.) following mandatory event 1 (e.g., a heart surgery), collect patient health data variable 1 through variable 20 following mandatory event 2 (e.g., a three month follow up), and collect patient health data variable 1 through variable 20 following mandatory event 3 (e.g., a six month follow up), with an optional collection endpoint of variable 1 through 20 for optional events 1 through n for complications and optional collection endpoint of variable 1 through 20 for optional events 1 through m for repeated procedures. The Clinical Study Patient Matching System 220 may receive, in this example, a data package from a healthcare provider system 140 that includes outcomes for mandatory event 1, a follow up event at 2.5 months named as a generic follow up event and a six month follow up event mislabeled as “mandatory event 4.” In the present example, “mandatory event 4” may include the expected variables properly tagged as variables 1 through 18, and may include tagged as variables 21 and 22, but which contain data expected as defined by the Clinical Study Patient Matching System 220 as variables 19 and 20. The Clinical Study Patient Matching System 220 may determine, based on the mapping of the stored data tags to the tags assigned by the study sponsor system, that the data tagged as variables 19 and 20 actually relate to the variable 19 and variable 20 tags defined by Clinical Study Patient Matching System 220. Accordingly, Clinical Study Patient Matching System 220 may reassign the proper data tag to variables 21 and 22, allowing the data to be compatibly shared between the study sponsor system 140.

In another example, the disclosed system is capable of simultaneously tracking current, up to date outcomes of patients selected for a study while simultaneously allowing a study sponsor to lock results for use in a study. According to various embodiments, the Clinical Study Patient Matching System 220 is configured to, upon receiving a selection of a subset of patient profiles for inclusion in a study from a study sponsor system 140, can create a copy of the selected data to be stored as part of an “study data store.” In a study data store, even when patients undergo additional medical procedures and additional data elements are collected and received by Clinical Study Patient Matching System 220, the study data store is locked and not updated after a particular patient has been included in a study. In contrast, the Clinical Study Patient Matching System 220 simultaneously, using methods well understood in the pertinent art, maintains and stores an “outcomes” database, in which the newly received and updated patient data is continuously included, thereby allowing the Clinical Study Patient Matching System 220 to independently monitor both study events and health outcomes that occur after a medical study has been completed.

In some examples, disclosed systems or methods may involve one or more of the following clauses:
Clause 1: A system comprising: one or more processors; and a non-transitory memory in communication with the one or more processors and storing instructions thereon, that when executed by the one or more processors, cause the system to: receive a first data package from a first healthcare provider system, the first data package comprising one or more first patient profiles comprising a plurality of data elements associated with a respective patient of the first healthcare provider system; assign to each data element a data tag indicating a data type for each data element; store the first data package and the data tags assigned to each data element of the first data package; receive, from a first user device, first study criteria comprising inclusion and exclusion criteria of a first study; map each of the inclusion and exclusion criteria of the first study to a respective data tag; based on the mapping and the data tags assigned to each data element, identify one or more second patient profiles from the first data package that are compatible with the inclusion and exclusion criteria of the first study; and share each data element associated with the identified one or more second patient profiles with the first user device.
Clause 2: The system of clause 1, wherein the non-transitory memory includes further instructions, that when executed by the one or more processors, are configured to cause the system to: receive, from the first user device, a selection of a subset of the one or more second patient profiles patient profiles comprising one or more third patient profiles; copy each data element associated with the one or more third patient profiles into a first study data store; and lock each data element of the first study data store.
Clause 3: The system of clause 2, wherein the non-transitory memory includes further instructions, that when executed by the one or more processors, are configured to cause the system to: copy each data element associated with the one or more third patient profiles into a first study outcomes database; determine that at least one data element associated with one or more third patient profiles has been modified or added; and update the first study outcomes database with the modified or added at least one data element.
Clause 4: The system of clause 1, wherein the non-transitory memory includes further instructions, that when executed by the one or more processors, are configured to cause the system to: receive a second data package from a second healthcare provider system, the second data package comprising: one or more fourth patient profiles comprising a plurality of data elements associated with a respective patient of the second healthcare provider system; and one or more pre-assigned data tags; wherein, each data element associated with the one or more fourth patient profiles is assigned one of the one or more pre-assigned data tags; determine that a first data element is improperly tagged with a first data tag of the one or more pre-assigned data tags; identify a second data tag of the one or more pre-assigned data tags that corresponds to the first data element; and assign the second data tag to the first data element.
Clause 5: The system of clause 1, wherein the non-transitory memory includes further instructions, that when executed by the one or more processors, are configured to cause the system to: receive, from a second user device, second study criteria comprising inclusion and exclusion criteria of a second study; map each of the inclusion and exclusion criteria of the second study to a respective data tag; based on the mapping and the data tags assigned to each data element, identify one or more fifth patient profiles from the first data package that are compatible with the inclusion and exclusion criteria of the second study; and share each data element associated with the identified one or more fifth patient profiles with the second user device.
Clause 6: The system of clause 1, wherein the non-transitory memory includes further instructions, that when executed by the one or more processors, are configured to cause the system to: receive, from a second user device, second study criteria comprising inclusion and exclusion criteria of a second study; map each of the inclusion and exclusion criteria of the second study to a respective data tag; based on the mapping and the data tags assigned to each data element, identify one or more sixth patient profiles from the first data package that are incompletely compatible with the inclusion and exclusion criteria of the second study; and transmit, to the first healthcare provider system, an indication of one or more additional data elements necessary for the one or more sixth patient profiles to be compatible with the inclusion and exclusion criteria of the second study.
Clause 7: The system of clause 1, wherein the non-transitory memory includes further instructions, that when executed by the one or more processors, are configured to cause the system to: receive, from the first user device, second study criteria comprising inclusion and exclusion criteria of a sub-study included within the first study; map each of the inclusion and exclusion criteria of the sub-study to a respective data tag; based on the mapping and the data tags assigned to each data element, identify one or more seventh patient profiles from the first data package that are compatible with the inclusion and exclusion criteria of the sub-study; and share each data element associated with the identified one or more seventh patient profiles with the first user device.
Clause 8: The system of clause 1, wherein assigning to each data element a data tag indicating a data type for each data element comprises comparing each data element to a stored data dictionary, wherein the stored data dictionary comprises an indication of an appropriate data tag for a plurality of data types.
Clause 9: The system of clause 8, wherein the non-transitory memory includes further instructions, that when executed by the one or more processors, are configured to cause the system to: responsive to determining that the stored data dictionary does not include the indication of the appropriate data tag for a respective data element of the first data package: generate a unique data tag; and apply the unique data tag to the respective data element.
Clause 10: A system comprising: one or more processors; and a non-transitory memory in communication with the one or more processors and storing instructions thereon, that when executed by the one or more processors, cause the system to: receive a first data package from a first healthcare provider system, the first data package comprising one or more first patient profiles comprising a plurality of data elements associated with a respective patient of the first healthcare provider system; receive a second data package from a second healthcare provider system, the second data package comprising one or more second patient profiles comprising a plurality of data elements associated with a respective patient of the second healthcare provider system; assign to each data element a data tag indicating a data type for each data element; store the first data package and the data tags assigned to each data element of the first data package; store the second data package and the data tags assigned to each data element of the second data package; receive, from a first user device, study criteria comprising inclusion and exclusion criteria for a first study; map each of the inclusion and exclusion criteria of the first study to a respective data tag; based on the mapping and the data tags assigned to each data element, identify one or more third patient profiles from the first data package and the second data package that are compatible with the inclusion and exclusion criteria of the first study; and share each data element associated with the identified one or more third patient profiles with the first user device.
Clause 11: The system of clause 10, wherein the non-transitory memory includes further instructions, that when executed by the one or more processors, are configured to cause the system to: receive, from a first user device, a selection of a subset of the one or more third patient profiles patient profiles comprising one or more fourth patient profiles; copy each data element associated with the one or more fourth patient profiles into a first study data store; and lock each data element of the first study data store.
Clause 12: The system of clause 11, wherein the non-transitory memory includes further instructions, that when executed by the one or more processors, are configured to cause the system to: copy each data element associated with the one or more fourth patient profiles into a first study outcomes database; determine that at least one data element associated with one or more fourth patient profiles has been modified or added; and update the first study outcomes database with the modified or added at least one data element.
Clause 13: The system of clause 10, wherein assigning to each data element a data tag indicating a data type for each data element comprises comparing each data element to a stored data dictionary, wherein the stored data dictionary comprises an indication of an appropriate data tag for a plurality of data types.
Clause 14: The system of clause 13, wherein the non-transitory memory includes further instructions, that when executed by the one or more processors, are configured to cause the system to: responsive to determining that the stored data dictionary does not include the indication of the appropriate data tag for a respective data element of the first data package: generate a unique data tag; and apply the unique data tag to the respective data element.
Clause 15: A system comprising: one or more processors; and a non-transitory memory in communication with the one or more processors and storing instructions thereon, that when executed by the one or more processors, cause the system to: receive a first data package from a first healthcare provider system, the first data package comprising one or more first patient profiles comprising a plurality of data elements associated with a respective patient of the first healthcare provider system, each of the plurality of data elements comprising an associated healthcare provider system tag; normalize the plurality of data elements by mapping each healthcare provider system tag to a predetermined compatibility data tag; assign to each data element the predetermined data tag indicating a data type for each data element identifying a data type for each data element; store the first data package and the data tags assigned to each data element of the first data package; receive, from a first user device, study criteria comprising inclusion and exclusion criteria for a first study; map each of the inclusion and exclusion criteria of the first study to a respective data tag; based on the mapping and the data tags assigned to each data element, identify one or more second patient profiles from the first data package that are compatible with the inclusion and exclusion criteria of the first study; and share each data element associated with the identified one or more second patient profiles with the first user device.
Clause 16: The system of clause 15, wherein the non-transitory memory includes further instructions, that when executed by the one or more processors, are configured to cause the system to: receive, from a first user device, a selection of a subset of the one or more second patient profiles patient profiles comprising one or more third patient profiles; copy each data element associated with the one or more third patient profiles into a first study data store; and lock each data element of the first study data store.
Clause 17: The system of clause 16, wherein the non-transitory memory includes further instructions, that when executed by the one or more processors, are configured to cause the system to: copy each data element associated with the one or more third patient profiles into a first study outcomes database; determine that at least one data element associated with one or more third patient profiles has been modified or added; and update the first study outcomes database with the modified or added at least one data element.
Clause 18: The system of clause 15, wherein assigning to each data element a data tag indicating a data type for each data element comprises comparing each data element to a stored data dictionary, wherein the stored data dictionary comprises an indication of an appropriate data tag for a plurality of data types.
Clause 19: The system of clause 18, wherein the non-transitory memory includes further instructions, that when executed by the one or more processors, are configured to cause the system to: responsive to determining that the stored data dictionary does not include the indication of the appropriate data tag for a respective data element of the first data package: generate a unique data tag; and apply the unique data tag to the respective data element.
Clause 20: The system of clause 15, wherein the non-transitory memory includes further instructions, that when executed by the one or more processors, are configured to cause the system to: receive, from the first user device, second study criteria comprising inclusion and exclusion criteria of a sub-study included within the first study; map each of the inclusion and exclusion criteria of the sub-study to a respective data tag; based on the mapping and the data tags assigned to each data element, identify one or more seventh patient profiles from the first data package that are compatible with the inclusion and exclusion criteria of the sub-study; and share each data element associated with the identified one or more seventh patient profiles with the first user device.

The features and other aspects and principles of the disclosed embodiments may be implemented in various environments. Such environments and related applications may be specifically constructed for performing the various processes and operations of the disclosed embodiments or they may include a general-purpose computer or computing platform selectively activated or reconfigured by program code to provide the necessary functionality. Further, the processes disclosed herein may be implemented by a suitable combination of hardware, software, and/or firmware. For example, the disclosed embodiments may implement general purpose machines configured to execute software programs that perform processes consistent with the disclosed embodiments. Alternatively, the disclosed embodiments may implement a specialized apparatus or system configured to execute software programs that perform processes consistent with the disclosed embodiments. Furthermore, although some disclosed embodiments may be implemented by general purpose machines as computer processing instructions, all or a portion of the functionality of the disclosed embodiments may be implemented instead in dedicated electronics hardware.

The disclosed embodiments also relate to tangible and non-transitory computer readable media that include program instructions or program code that, when executed by one or more processors, perform one or more computer-implemented operations. The program instructions or program code may include specially designed and constructed instructions or code, and/or instructions and code well-known and available to those having ordinary skill in the computer software arts. For example, the disclosed embodiments may execute high level and/or low-level software instructions, such as machine code (e.g., such as that produced by a compiler) and/or high-level code that can be executed by a processor using an interpreter.

The technology disclosed herein typically involves a high-level design effort to construct a computational system that can appropriately process unpredictable data. Mathematical algorithms may be used as building blocks for a framework, however certain implementations of the system may autonomously learn their own operation parameters, achieving better results, higher accuracy, fewer errors, fewer crashes, and greater speed.

As used in this application, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

As an example, embodiments or implementations of the disclosed technology may provide for a computer program product, including a computer-usable medium having a computer-readable program code or program instructions embodied therein, said computer-readable program code adapted to be executed to implement one or more functions specified in the flow diagram block or blocks. Likewise, the computer program instructions may be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

Certain implementations of the disclosed technology described above with reference to user devices may include mobile computing devices. Those skilled in the art recognize that there are several categories of mobile devices, generally known as portable computing devices that can run on batteries but are not usually classified as laptops. For example, mobile devices can include, but are not limited to portable computers, tablet PCs, internet tablets, PDAs, ultra-mobile PCs (UMPCs), wearable devices, and smart phones. Additionally, implementations of the disclosed technology can be utilized with internet of things (IoT) devices, smart televisions and media devices, appliances, automobiles, toys, and voice command devices, along with peripherals that interface with these devices.

In this description, numerous specific details have been set forth. It is to be understood, however, that implementations of the disclosed technology may be practiced without these specific details. In other instances, well-known methods, structures, and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form. By “comprising” or “containing” or “including” is meant that at least the named element, or method step is present in article or method, but does not exclude the presence of other elements or method steps, even if the other such elements or method steps have the same function as what is named.

It is to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Although embodiments are described herein with respect to systems or methods, it is contemplated that embodiments with identical or substantially similar features may alternatively be implemented as systems, methods and/or non-transitory computer-readable media.

As used herein, unless otherwise specified, the use of the ordinal adjectives “first,” “second,” “third,” etc., to describe a common object, merely indicates that different instances of like objects are being referred to, and is not intended to imply that the objects so described must be in a given sequence, either temporally, spatially, in ranking, or in any other manner.

While certain embodiments of this disclosure have been described in connection with what is presently considered to be the most practical and various embodiments, it is to be understood that this disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the technology and also to enable any person skilled in the art to practice certain embodiments of this technology, including making and using any apparatuses or systems and performing any incorporated methods. The patentable scope of certain embodiments of the technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system comprising:
one or more processors; and
a non-transitory memory in communication with the one or more processors and storing instructions thereon, that when executed by the one or more processors, cause the system to:
receive a first data package from a first healthcare provider system, the first data package comprising one or more first patient profiles comprising a plurality of data elements associated with a respective patient of the first healthcare provider system;
assign to each data element a data tag indicating a data type for each data element;
store the first data package and the data tags assigned to each data element of the first data package;
receive, from a first user device, first study criteria comprising inclusion and exclusion criteria of a first study;
map each of the inclusion and exclusion criteria of the first study to a respective data tag;
based on the mapping and the data tags assigned to each data element, identify one or more second patient profiles from the first data package that are compatible with the inclusion and exclusion criteria of the first study; and
share each data element associated with the identified one or more second patient profiles with the first user device.

2. The system of claim 1, wherein the non-transitory memory includes further instructions, that when executed by the one or more processors, are configured to cause the system to:
receive, from the first user device, a selection of a subset of the one or more second patient profiles patient profiles comprising one or more third patient profiles;
copy each data element associated with the one or more third patient profiles into a first study data store; and
lock each data element of the first study data store.

3. The system of claim 2, wherein the non-transitory memory includes further instructions, that when executed by the one or more processors, are configured to cause the system to:
copy each data element associated with the one or more third patient profiles into a first study outcomes database;
determine that at least one data element associated with one or more third patient profiles has been modified or added; and
update the first study outcomes database with the modified or added at least one data element.

4. The system of claim 1, wherein the non-transitory memory includes further instructions, that when executed by the one or more processors, are configured to cause the system to:
receive a second data package from a second healthcare provider system, the second data package comprising:
one or more fourth patient profiles comprising a plurality of data elements associated with a respective patient of the second healthcare provider system; and
one or more pre-assigned data tags;
wherein, each data element associated with the one or more fourth patient profiles is assigned one of the one or more pre-assigned data tags;
determine that a first data element is improperly tagged with a first data tag of the one or more pre-assigned data tags;
identify a second data tag of the one or more pre-assigned data tags that corresponds to the first data element; and
assign the second data tag to the first data element.

5. The system of claim 1, wherein the non-transitory memory includes further instructions, that when executed by the one or more processors, are configured to cause the system to:
receive, from a second user device, second study criteria comprising inclusion and exclusion criteria of a second study;
map each of the inclusion and exclusion criteria of the second study to a respective data tag;
based on the mapping and the data tags assigned to each data element, identify one or more fifth patient profiles from the first data package that are compatible with the inclusion and exclusion criteria of the second study; and
share each data element associated with the identified one or more fifth patient profiles with the second user device.

6. The system of claim 1, wherein the non-transitory memory includes further instructions, that when executed by the one or more processors, are configured to cause the system to:
receive, from a second user device, second study criteria comprising inclusion and exclusion criteria of a second study;
map each of the inclusion and exclusion criteria of the second study to a respective data tag;
based on the mapping and the data tags assigned to each data element, identify one or more sixth patient profiles from the first data package that are incompletely compatible with the inclusion and exclusion criteria of the second study; and
transmit, to the first healthcare provider system, an indication of one or more additional data elements necessary for the one or more sixth patient profiles to be compatible with the inclusion and exclusion criteria of the second study.

7. The system of claim 1, wherein the non-transitory memory includes further instructions, that when executed by the one or more processors, are configured to cause the system to:
receive, from the first user device, second study criteria comprising inclusion and exclusion criteria of a sub-study included within the first study;
map each of the inclusion and exclusion criteria of the sub-study to a respective data tag;
based on the mapping and the data tags assigned to each data element, identify one or more seventh patient profiles from the first data package that are compatible with the inclusion and exclusion criteria of the sub-study; and
share each data element associated with the identified one or more seventh patient profiles with the first user device.

8. The system of claim 1, wherein assigning to each data element a data tag indicating a data type for each data element comprises comparing each data element to a stored data dictionary, wherein the stored data dictionary comprises an indication of an appropriate data tag for a plurality of data types.

9. The system of claim 8, wherein the non-transitory memory includes further instructions, that when executed by the one or more processors, are configured to cause the system to:
responsive to determining that the stored data dictionary does not include the indication of the appropriate data tag for a respective data element of the first data package:
generate a unique data tag; and
apply the unique data tag to the respective data element.

10. A system comprising:
one or more processors; and
a non-transitory memory in communication with the one or more processors and storing instructions thereon, that when executed by the one or more processors, cause the system to:
receive a first data package from a first healthcare provider system, the first data package comprising one or more first patient profiles comprising a plurality of data elements associated with a respective patient of the first healthcare provider system;
receive a second data package from a second healthcare provider system, the second data package comprising one or more second patient profiles comprising a plurality of data elements associated with a respective patient of the second healthcare provider system;
assign to each data element a data tag indicating a data type for each data element;
store the first data package and the data tags assigned to each data element of the first data package;
store the second data package and the data tags assigned to each data element of the second data package;
receive, from a first user device, study criteria comprising inclusion and exclusion criteria for a first study;
map each of the inclusion and exclusion criteria of the first study to a respective data tag;
based on the mapping and the data tags assigned to each data element, identify one or more third patient profiles from the first data package and the second data package that are compatible with the inclusion and exclusion criteria of the first study; and
share each data element associated with the identified one or more third patient profiles with the first user device.

11. The system of claim 10, wherein the non-transitory memory includes further instructions, that when executed by the one or more processors, are configured to cause the system to:
receive, from a first user device, a selection of a subset of the one or more third patient profiles patient profiles comprising one or more fourth patient profiles;
copy each data element associated with the one or more fourth patient profiles into a first study data store; and
lock each data element of the first study data store.

12. The system of claim 11, wherein the non-transitory memory includes further instructions, that when executed by the one or more processors, are configured to cause the system to:
copy each data element associated with the one or more fourth patient profiles into a first study outcomes database;
determine that at least one data element associated with one or more fourth patient profiles has been modified or added; and
update the first study outcomes database with the modified or added at least one data element.

13. The system of claim 10, wherein assigning to each data element a data tag indicating a data type for each data element comprises comparing each data element to a stored data dictionary, wherein the stored data dictionary comprises an indication of an appropriate data tag for a plurality of data types.

14. The system of claim 13, wherein the non-transitory memory includes further instructions, that when executed by the one or more processors, are configured to cause the system to:
responsive to determining that the stored data dictionary does not include the indication of the appropriate data tag for a respective data element of the first data package:
generate a unique data tag; and
apply the unique data tag to the respective data element.

15. A system comprising:
one or more processors; and
a non-transitory memory in communication with the one or more processors and storing instructions thereon, that when executed by the one or more processors, cause the system to:
receive a first data package from a first healthcare provider system, the first data package comprising one or more first patient profiles comprising a plurality of data elements associated with a respective patient of the first healthcare provider system, each of the plurality of data elements comprising an associated healthcare provider system tag;
normalize the plurality of data elements by mapping each healthcare provider system tag to a predetermined compatibility data tag;
assign to each data element the predetermined data tag indicating a data type for each data element identifying a data type for each data element;
store the first data package and the data tags assigned to each data element of the first data package;
receive, from a first user device, study criteria comprising inclusion and exclusion criteria for a first study;
map each of the inclusion and exclusion criteria of the first study to a respective data tag;
based on the mapping and the data tags assigned to each data element, identify one or more second patient profiles from the first data package that are compatible with the inclusion and exclusion criteria of the first study; and
share each data element associated with the identified one or more second patient profiles with the first user device.

16. The system of claim 15, wherein the non-transitory memory includes further instructions, that when executed by the one or more processors, are configured to cause the system to:
receive, from a first user device, a selection of a subset of the one or more second patient profiles patient profiles comprising one or more third patient profiles;
copy each data element associated with the one or more third patient profiles into a first study data store; and
lock each data element of the first study data store.

17. The system of claim 16, wherein the non-transitory memory includes further instructions, that when executed by the one or more processors, are configured to cause the system to:
copy each data element associated with the one or more third patient profiles into a first study outcomes database;
determine that at least one data element associated with one or more third patient profiles has been modified or added; and
update the first study outcomes database with the modified or added at least one data element.

18. The system of claim 15, wherein assigning to each data element a data tag indicating a data type for each data element comprises comparing each data element to a stored data dictionary, wherein the stored data dictionary comprises an indication of an appropriate data tag for a plurality of data types.

19. The system of claim 18, wherein the non-transitory memory includes further instructions, that when executed by the one or more processors, are configured to cause the system to:
responsive to determining that the stored data dictionary does not include the indication of the appropriate data tag for a respective data element of the first data package:
generate a unique data tag; and
apply the unique data tag to the respective data element.

20. The system of claim 15, wherein the non-transitory memory includes further instructions, that when executed by the one or more processors, are configured to cause the system to:
receive, from the first user device, second study criteria comprising inclusion and exclusion criteria of a sub-study included within the first study;
map each of the inclusion and exclusion criteria of the sub-study to a respective data tag;
based on the mapping and the data tags assigned to each data element, identify one or more seventh patient profiles from the first data package that are compatible with the inclusion and exclusion criteria of the sub-study; and
share each data element associated with the identified one or more seventh patient profiles with the first user device.
